(19) United States
(12) Patent Application Publication
(10) Pub. No.: US 2017/0282224 A1
(43) Pub. Date: Oct. 5, 2017

(54) INDIVIDUAL VESSEL UTILIZED FOR CLEANING, LUBRICATING AND STERILIZING DEVICES FOR HEAT STERILIZER

(71) Applicants: Thad J. Overmyer, Danville, KY (US); MICHAEL OVERMYER, DANVILLE, KY (US)

(72) Inventors: Thad J. Overmyer, Danville, KY (US); MICHAEL OVERMYER, DANVILLE, KY (US)

(21) Appl. No.: 15/473,233

(22) Filed: Mar. 29, 2017

Related U.S. Application Data
(60) Provisional application No. 62/390,454, filed on Mar. 30, 2016.

(51) Int. Cl. B08B 9/027; A61L 2/04; F16N 19/00; A61L 2/26

(52) U.S. Cl. CPC B08B 9/027; A61L 2/26; A61L 2/04; F16N 19/00; A61L 2202/24; A61L 2202/14

(57) ABSTRACT

A two-piece sterilizable vessel is sealed together containing devices and a solution that cleans and/or lubricates devices during heat sterilization. When placed inside a heat sterilizer, the vessel becomes pressurized and isolates the solution from the sterilizer's environment. This vessel has two parts: a base and a top.

The base has an internal calibrated reservoir to measure the amount of solution within the vessel.

The base's external dimensions, larger than the top, creates a lengthwise tilt of the vessel when placed on a flat surface. The top is positioned lower than the base. The purpose of the tilt is to allow the solution to flow from the reservoir to the vessel's top. This solution immerses the mechanics of the device.

After sterilization is completed, the pressure relief valve in the vessel's top is activated to release the pressure within the vessel.

### CROSS REFERENCE TO RELATED APPLICATION

This non-provisional application is a continuation-in-part of my co-pending provisional application titled, “Container for Autoclave” filed on Mar. 30, 2016 and given Ser. No. 62/390,454.

### BACKGROUND OF THE INVENTION

Devices used in the medical and dental fields for procedures require cleaning, lubrication and sterilization. The heat sterilization process of a device affects the life span of the device. When the device is exposed to body fluids or sources of contaminates, the device requires cleaning and possibly lubrication of the device prior to sterilization. A device with moving parts (internal or external) are cleaned and lubricated prior to sterilization.

When a device is not cleaned properly, contaminates remaining on the device will create a build up of sterilized layers of contaminates. This build up of contaminates may affect the function, sterilization efficiency and the life span of the device. The lubrication and cleaning products should not interfere with the sterilizing process or the mechanics of the heat sterilizer.

Prior to steam sterilization, water immiscible based products are used to clean or lubricate the devices with air and water conduits. These products can affect the flow of the steam and therefore prevent the proper sterilization process.

The following devices in the dental and medical fields with moving parts that need proper cleaning and lubrication are the following: contra angle, laser, handpiece (slow and high speed), hinged device, and all surgical devices or any device that contacts the body internally or externally.

All of these devices are exposed to contamination such as the patient's body fluids, saliva, blood, tooth structure particles, restorative material particles, material for cleaning teeth, bone spicules, polishing burrs containing grit and rubber particles. When the contra angle is exposed to contaminates, this allows the internal gears or bearing mechanisms of these angles to be contaminated.

Presently, the three known methods of cleaning and lubricating the contaminated contra angle prior to sterilization are the following:
1.Apply a lubricant product into the head of the contra angle.
2. Immerse the head of the contra angle in a cleaning solution and then immerse the head of the contra angle in a lubricating solution.
3. Take the contra angle apart, which then is cleaned and lubricated.
   After the cleaning and lubricating process is performed, then the contra angle is bagged for sterilization.

The high-speed handpiece operates at speeds up to 500,000 RPM's. Although the handpiece parts such as O-rings, bearings, impeller or turbine are enclosed in a canister type housing, the high-speed rotation of the impeller or the turbine creates a centrifugal force to cause contaminates to be brought into these internal handpiece parts. Over a period of time without proper cleaning and lubrication of these devices, the life span of the device's moving parts are affected by the build up of contaminates and the heat sterilizing of these contaminates. Materials such as oral fluids, blood, tooth structure, filling material structures, or bone spicules sucked into the internal parts lead to failure of the handpiece. These materials can be detrimental to the life span of the turbine.

The air-driven handpiece has four conduits. Two large conduits are for the air-drive to the turbine and for the exhaust. Two small conduits focus at the handpiece head (operative end). One small conduit is a water outlet. The other small conduit is an air outlet. The pressurized air and water from the two small conduits create a spray directed upon the work piece. This spray directed upon the pressurized air exiting from the air-drive of the handpiece increases the aerosol. The aerosol can carry contamination into the air of the operatory.

The handpiece is cleaned by manual or automated devices. These devices introduce an air source into the air drive conduit of the handpiece. This pressurized air forces the contaminates from the internal parts of the handpiece. For lubrication of the handpiece, an oily product is introduced into the drive airline to lubricate bearings and the turbine by a pressurized air method. The two small conduits (air and water outlets) and the exhaust conduit are not always cleaned and lubricated. The handpiece is placed in a paper sterilization bag or plastic vessel and sterilized.

The lubricant within the handpiece tubing (drive line) may impede the penetration of the steam from the heat sterilizer, as the lubricant and steam are not miscible. This creates improper sterilization of the area. The lubricant becomes gooey with the mixture of steam during sterilization. This gooey material builds up within the handpiece's internal parts and affects the life span of the handpiece. The waterline to the head of the handpiece is untreated and any particles surrounding the orifice of the water outlet and bacteria within the waterline are baked on the tubing. The automated cleaning lubrication device may fail without the operator's knowledge.

The laser has a waterline used to cool the workpiece and/or the device during procedures. Maintaining a clean waterline is important to prevent contamination of the work area and aerosols.

The heat sterilizer's cleanliness is important to maintain function and effectiveness. Oil vapors and overheated paper remnants from sterilization bags have an affect upon the performance of the heat sterilizer.

### SUMMARY OF THE INVENTION

A two-piece sealed heat sterilizable vessel is designed to contain devices and a solution that cleans and/or lubricates devices during heat sterilization. The vessel's design may be any geometric shape, but should have a lengthwise tilt when placed on a flat surface. This vessel has two parts: a base and a top.

The base has a larger external dimension than the top. The difference in this dimension creates a lengthwise tilt of the vessel. When the vessel is laid onto a flat surface inside the heat sterilizer, the vessel's top is positioned lower compared to the vessel's base. The purpose of the vessel's tilt is to allow the solution inside the vessel to immerse the mechanics of the device that is also inside the vessel.

The vessel's base has female threads. The vessel's base has an internal reservoir. The reservoir has calibrated chambers to contain a measured amount of solution. The chamber's opening is less than the device's size so the device does not enter the chamber. The solution poured into the reservoir will be a measured amount relative to the size and number of the chambers.

A shoulder within the vessel's base is positioned directly between the female threads and the orifices of the reservoir's chambers. This shoulder has an undercut into the walls of the vessel's base to hold a flat high heat resistant washer in place. This washer is necessary to seal the base and the top when the two parts of the vessel are screwed together.

The vessel's top has male threads with a pressure relief valve within the end, opposite the threaded end.

The device's head is placed in the vessel's top with the head of the device opposite of the base's reservoir. The two-piece sealed heat sterilizable vessel is screwed together tightly and placed inside the heat sterilizer.

The base of the vessel with a larger dimension tilts the vessel at an angle when placed on a flat surface. The solution flows to the top of the vessel allowing the solution to cover the device's mechanics (internal parts). The vessel is designed to allow the device's mechanics to be lower than the rest of the body of the device and immersed in the lubricating/cleaning solution when the vessel is placed on a flat surface inside the heat sterilizer.

During sterilization, the heat sterilizer builds up pressure and heat causing the sealed vessel to become pressurized, and no solution and vapors will be emitted from the sealed vessel. The lubricating/cleaning solution begins to percolate and vaporize through the internal parts and the conduits of the device. The solution's vapors then passes through the conduits within the device. The solution is water miscible and penetrates the remaining water within the conduits.

After the heat sterilizer's cycle has been completed, the vessel is removed and placed on its base allowing the solution to drain into the base's reservoir away from the device's mechanics. The heat from the sterilizable vessel will help dry the device, which is now positioned out of the solution.

The vessel's top has a pressure relief valve that releases the pressure within the vessel. Once the vessel is cooled and the internal pressure is released, the vessel may be opened.

### BRIEF DESCRIPTION OF DRAWINGS

The drawings are views of the external and internal parts of the vessel.

FIG. 1 is the external view of the two-piece vessel; and

FIG. 2 is the internal view depicting the vessel; and

FIG. 3 is the internal view of the top portion of vessel; and

FIG. 4 is the end section of the top piece of a threaded conduit; and

FIG. 5 is the internal view of the base of the vessel; and

FIG. 6 is the internal view of the base of vessel showing the reservoir's chambers.

### DESCRIPTION OF THE PREFERRED EMBODIMENT

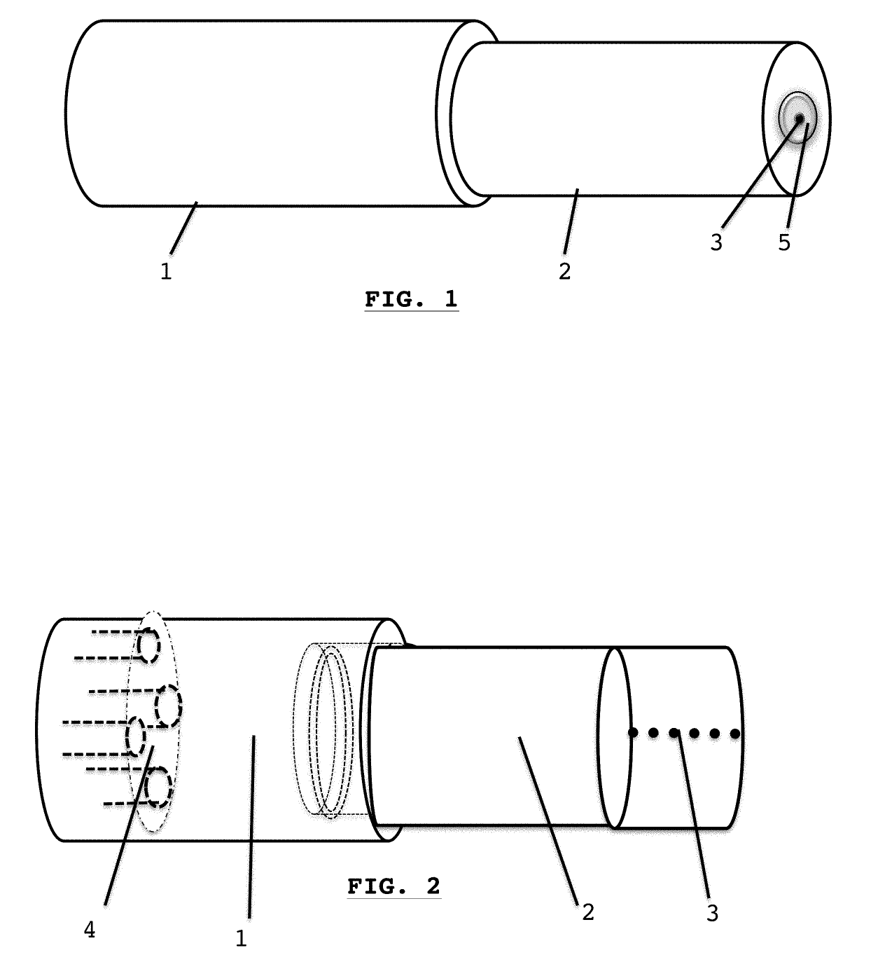

FIG. 1 illustrates a two-piece cylinder shaped vessel: the base 1 and the top 2 of the vessel. The end of the top 2 has a depressed area 5 with an opening from the conduit 3. The base 1 is larger in dimension than the top 2. This vessel will be used to contain a solution and devices that need cleaning, lubricating and sterilizing.

FIG. 2 shows the internal view of the vessel. The base 1 has female threads and the top 2 has male threads. A reservoir 4 is within the base 1. The top 2 has a threaded conduit 3 which will hold a pressure relief valve.

FIG. 3 depicts a partial cutaway of the top 2 showing the male threads 6 with the internal chamber 10. The threaded conduit 3 connects the chamber 10 to the atmosphere.

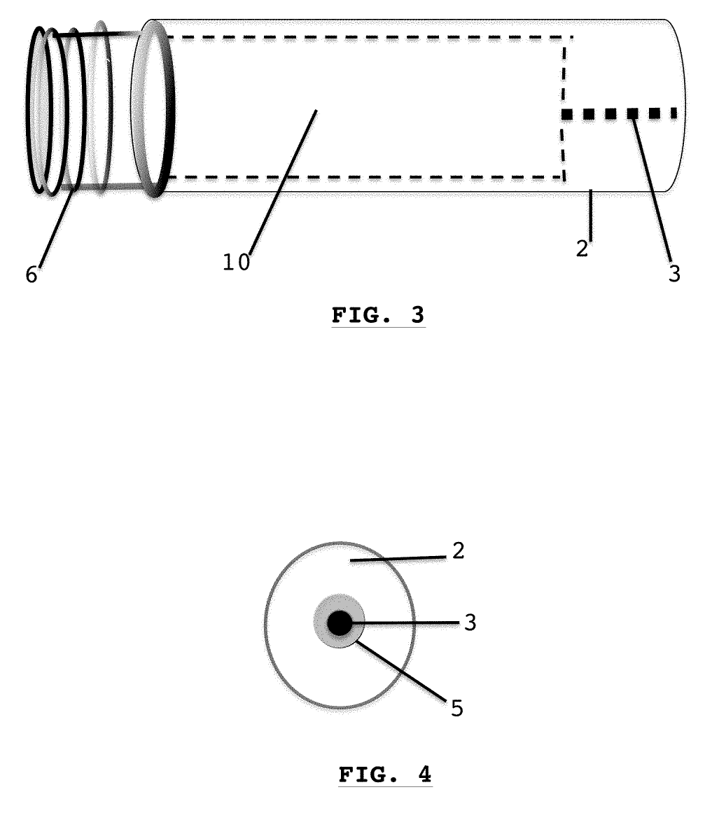

FIG. 4 shows the end opening of conduit 3 and the circular depressed area 5 at the end of the top 2.

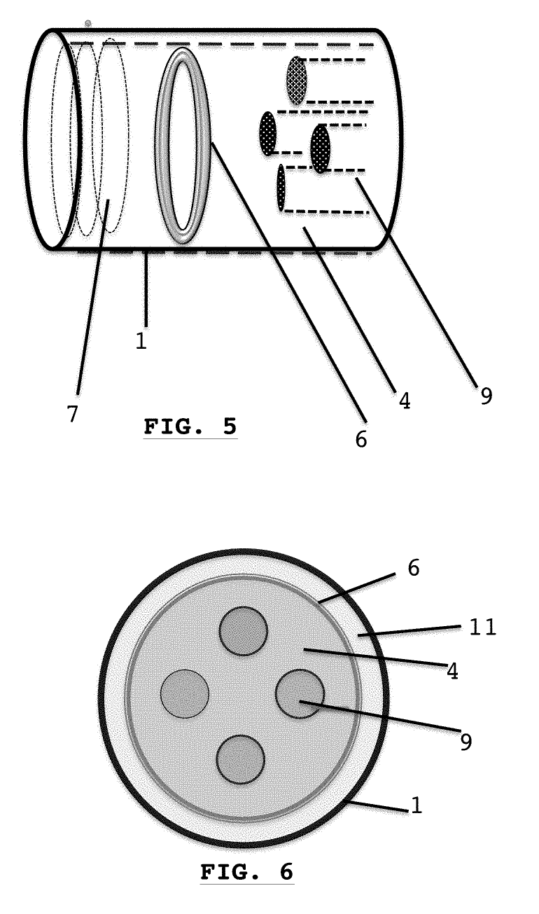

FIG. 5 depicts the internal side view of the base 1 and the area of the reservoir 4. The female threads 7 of base 1 and the reservoir 4 are shown. A shoulder 6 within the vessel's base is positioned directly between the female threads and the orifices of the reservoir 4. This shoulder 6 contains a washer that allows the vessel to be sealed when base 1 and top 2 are screwed together. The reservoir 4 consists of chambers 9. The measured volume of solution needed for the process is dictated by the chambers' 9 volume. The chambers 9 are placed at the end of the base 1 to allow the solution to be measured and drained back into the reservoir 4 at the end of sterilization. The cross-section dimension of the chambers 9 should be less than the dimension of the device to be placed in the vessel. This will allow the device to remain out of the reservoir's solution after sterilization.

FIG. 6 shows the internal view of the base 1 of vessel depicting the reservoir's chambers 9, the shoulder 6 for the washer and the thickness 11 of the base 1.

A tilt of the vessel occurs when laid on a flat surface inside the heat sterilizer. This tilt occurs since the base is larger in dimension than the top. When a solution is placed inside the vessel and placed on a flat surface, the solution will drain into the top 2 as the vessel will be on a tilt. The solution will cover the part of the device that requires cleaning, lubricating and sterilizing during sterilization.

After sterilization is complete, the vessel is placed on its base 1 to allow the solution to drain back into the chambers 9 of the reservoir 4. The vessel's heat from sterilization will allow the device to dry within the vessel.

When the vessel is cool, the pressure relief valve in the conduit 3 can be depressed to release the vessel's internal pressure. The base 1 and top 2 of the two-piece vessel can be unscrewed.

I claim:

1. A two-piece sealed vessel containing devices and a solution to clean and/or lubricate devices during heat sterilization. The one piece being of a larger dimension than the other piece would have a reservoir opposite end of the threaded opening and the other piece would have a threaded conduit opposite end of the threaded end.
a. The two pieces of different dimensions allows a lengthwise tilt in the vessel when placed on a flat surface.
b. The tilt will allow the solution to flow from the reservoir to the other end to immerse the mechanics of the devices within the vessel.
c. The one piece with the reservoir would have multiple chambers.
i. The cross-section dimension of the chambers should be less than the dimension of the device to be placed in the vessel. This will allow the device to remain out of the reservoir's solution.
ii. A measuring method would be the number and dimensions of the chambers, which would calibrate the amount of fluid to be used in the vessel.
iii. A ledge within the wall of the vessel between the threads and the reservoir would maintain a washer to seal the two pieces when screwed together.
iv. The piece with the reservoir would have female threads.
d. The other piece with the threaded conduit would have male threads.
i. The threaded conduit would hold a pressure relief valve.

2. A sealed vessel isolates the solution from the heat sterilizer's environment as the solution boils and vaporizes the device within the vessel during heat sterilization.
a. During sterilization, the heat sterilizer builds up pressure and heat causing the sealed vessel to become pressurized, and no solution and vapors will be emitted from the sealed vessel.
i. The solution will cover the part of the device that requires cleaning, lubricating and sterilizing during sterilization.

3. The vessel is made of material to withstand pressure and the temperature of heat sterilization.
